(19) United States
(12) Patent Application Publication
(10) Pub. No.: US 2013/0131318 A1
(43) Pub. Date: May 23, 2013

(54) SINGLE UNIT ANTIBODY PURIFICATION

(71) Applicants: Diderik Reinder Kremer, Geleen (NL); Randal William Maarleveld, Geleen (NL)

(72) Inventors: Diderik Reinder Kremer, Geleen (NL); Randal William Maarleveld, Geleen (NL)

(21) Appl. No.: 13/578,679

(22) PCT Filed: Feb. 10, 2011
(86) PCT No.: PCT/EP2011/051975

§ 371 Date: Oct. 31, 2012

(30) Foreign Application Priority Data
Feb. 12, 2010 (EP) 10153529.2

(51) Int. Cl. B01D 15/32; C07K 16/00

(52) U.S. Cl. CPC B01D 15/32; C07K 16/00
USPC 530/387.1; 210/198.2

(57) ABSTRACT

The present invention relates to a method for the purification of antibodies from a protein mixture produced in a bioreactor, at least comprising the steps of intermediate purification and polishing, wherein the intermediate purification and polishing step comprises in-line anion exchange chromatography (AEX) treatment and hydrophobic interaction chromatography (HIC) treatment in flow through mode. The present invention further relates to a single operational unit comprising both an anion exchange chromatography part and a hydrophobic interaction chromatography part, which are serially connected, wherein the unit comprises an inlet at the upstream end of the anion exchange chromatography part and an outlet at the downstream end of the hydrophobic interaction chromatography part and wherein the unit also comprises an inlet between the anion exchange chromatography part and the hydrophobic interaction chromatography part.

The present invention relates to a method for single unit purification of antibodies and to equipment which can be used in this method.

The purification of monoclonal antibodies, produced by cell culture, for use in pharmaceutical applications is a process involving a large number of steps. The antibodies are essentially to be freed from all potentially harmful contaminants such as proteins and DNA originating from the cells producing the antibodies, medium components such as insulin, PEG ethers and antifoam as well as any potentially present infectious agents such as viruses and prions.

Typical processes for purification of antibodies from a culture of cells producing these proteins are described in BioPharm International Jun. 1, 2005, Downstream Processing of Monoclonal Antibodies: from High Dilution to High Purity.

As antibodies are produced by cells, such as hybridoma cells or transformed host cells (like Chinese Hamster Ovary (CHO) cells, mouse myeloma-derived NS0 cells, Baby Hamster Kidney (BHK) cells and human retina-derived PER.C6® cells), the particulate cell material will have to be removed from the cell broth, preferably early in the purification process. This part of the process is indicated here as “clarification”. Subsequently or as part of the clarification step the antibodies are purified roughly to at least about 80%, usually with a binding plus eluting chromatography step (in the case of IgG often using immobilized Protein A). This step, indicated here as “capturing” not only results in a first considerable purification of the antibody, but may also result in a considerable reduction of the volume, hence concentration of the product. Alternative methods for capturing are for example Expanded Bed Adsorption (EBA), 2-phase liquid separation (using e.g. polyethyleneglycol) or fractionated precipitation with lyotropic salt (such as ammonium sulfate).

Subsequent to clarification and capturing, the antibodies are further purified. Generally, at least 2 chromatographic steps are required after capturing to sufficiently remove the residual impurities. The chromatographic step following capturing is often called intermediate purification step and the final chromatographic step generally is called the polishing step. Each of these steps is generally performed as single unit operation in batch mode and at least one of these steps is carried out in the binding plus eluting mode. In addition, each chromatographic step requires specific loading conditions with respect to e.g. pH, conductivity etc. Therefore, extra handling has to be performed prior to each chromatography step in order to adjust the load to the required conditions. All of this mentioned makes the process elaborate and time consuming. The impurities generally substantially removed during these steps are process derived contaminants, such as host cell proteins, host cell nucleic acids, culture medium components (if present), protein A (if present), endotoxin (if present), and micro-organisms (if present).

Many methods for such purification of antibodies have been described in the prior art:
WO2007/076032 describes a method for the purification of antibodies (CTLA4-Ig and variants thereof) wherein a cell culture the supernatant or a fraction thereof obtained after affinity chromatography is subjected to anion exchange chromatography to obtain an eluted protein product and the eluted protein product is subjected to hydrophobic interaction chromatography so as to obtain an enriched protein product. In this process the eluted protein product” is obtained by a process wherein the antibodies are first captured to the anion exchange chromatography material, the exchange chromatography material is subsequently washed with a wash buffer whereafter the antibodies are eluted therefrom by changing of the process conditions (eluting with an elution buffer).
US2008/0167450 relates to the purification of Fc containing proteins such as antibodies by binding the proteins to a protein A column and eluting with a pH gradient elution system. This document describes the desirability to apply hydrophobic interaction chromatography and anion exchange chromatography in flow-through mode [par 0058-0064).
WO2008/025747 relates to the purification of Fc-fusion proteins in a process comprising protein A or G chromatography, cation exchange chromatography, anion exchange chromatography and hydroxyapatite chromatography employed specifically in this order. In this process both the anion exchange chromatography and the hydroxyapatite chromatography are applied in flow-through mode.
US2007/0167612 is concerned with purification of proteins such as antibodies which are first captured to an affinity column, like a protein A column. The eluate from the affinity column is subsequently contacted with anion exchange material to which the antibodies bind and subsequently are eluted. For the further purification additional chromatography columns and purification steps may be employed, including additional cation-exchange chromatography, anion-exchange chromatography, size exclusion chromatography, affinity chromatography, hydroxyapatite chromatography, and hydrophobic interaction chromatography.
WO2001/072769 describes the purification of highly anionic proteins, for example sulfated proteins. To this end subsequent anion exchange and hydrophobic interaction chromatography were used, both in bind-and-elute mode.
WO2009/058769 relates to methods of removing impurities from antibody preparation. In particular it relates to a method of purifying antibodies containing hydrophobic variants. To this end a sample is loaded on a Protein A column; eluted from the Protein A column with a proper eluting solution, loaded on an cation and or anion exchange column; eluted from this ion exchange column, loaded on a hydrophobic interaction chromatography (HIC) column, wherein the HIC column is in a flow through mode whereafter the purified material is collected. Note that only the HIC column is applied in flow-through mode.
EP1614694 deals with purification and separation of immunoglobulins. In particular it deals with purification of antibodies from a cell culture in subsequent protein A, anion exchange and cation exchange column steps, optionally followed by a hydrophobic interaction column step. Of these steps the anion exchange column step is operated in flow-through, all other steps in bind-and-elute mode.
WO2008/051448 relates to reducing protein A contamination in antibody preparations which are purified using protein A affinity chromatography. It is been suggested that this protein A contamination can be removed using a charge modified depth filter. This removal step can be preceded by or followed by purification steps conventional for antibody preparations.
EP0530447 describes antibody purification by anion, cation and hydrophobic interaction chromatography combined with a specific sterilization step. The order of the chromatographic steps may vary. Each of the chromatographic steps is operated in bind-and-elute mode.
Kuczewski, M. et al. (2009) [Biotechn. Bioengn. 105, 296-305]. Describes the use of hydrophobic interaction membrane absorbers for the polishing of antibodies.
Chen, J. et al. (2008) [J. Chrom. A 1177, 272-281]. Comparison of conventional and new generation hydrophobic interaction chromatography resins (like mixed mode) in the purification of antibodies.
Zhou, J. X. et al. (2006) [J. Chrom. A 1134 66-73]. Describes use of hydrophobic interaction membrane absorbers as alternative to hydrophobic interaction column chromatography.
Gottschalk, U. (2008) [Biotechnol. Prog. 24, 496-503]. Discusses the disadvantages of column chromatography in antibody purification over the use of membrane adsorbers.
Wang, C. et al. (2007) [J. Chrom. A 1155, 74-84]. Use of cored anion-exchange chromatography in a flow-through process for the removal of trace contaminations (polishing) from antibody material. Comparison with non-cored anion exchange material.
Azevedo, A. et al. (2008) [J. Chrom. A. 1213, 154-161]. Integrated process for the purification of antibodies combining aqueous two-phase extraction, hydrophobic interaction chromatography and size-exclusion chromatography.
Boi, C. (2007) [J. Chrom. B. 848, 19-27]. This review considers the use of membrane adsorbers as an alternative technology for capture and polishing steps for the purification of monoclonal antibodies.

Disadvantages of the methods described above are long operation times, high variable costs (for example due to the necessity of large column capacity, which is inherently required for a binding plus eluting step, and hence large amounts of costly resins needed) and high fixed cost (due to labor costs).

According to the present invention, very efficient removal of residual impurities from cell culture-produced antibodies can be achieved by using serial, in-line anion exchange chromatography (AEX) and hydrophobic interaction chromatography (HIC) both in the flow-through mode and preferably operating as one single unit operation. In-line mixing of a lyotropic salt after the AEX and before the HIC can be used to adjust the right conditions for the hydrophobic interaction chromatography.

Advantages of this process with separate serially connected in-line AEX and HIC devices both used in flow-through mode are considerable reduction of the operation time and labor and lower operational costs. In addition, smaller (and thus less costly) chromatographic units are required, since all units operate in flow through mode which requires only sufficient binding capacity for the impurities and not for the product.

Therefore, the present invention can be defined as a method for the purification of antibodies from a cell broth produced in a bioreactor, at least comprising the steps of intermediate purification and polishing, wherein the novel purification step comprises serial in-line anion exchange chromatography (AEX) treatment yielding as a flow through fraction a separation mixture followed by hydrophobic interaction chromatography (HIC) treatment yielding as a flow-through fraction a purified antibody preparation, and wherein the purified antibody preparation is subjected to at least one further purification step.

In the context of the present invention, the “separation mixture” is the solution resulting from the first ion exchange step according to the invention, and the “purified antibody preparation” is the solution resulting from the second ion exchange step according to the invention. It is intended to adhere to this terminology throughout the present application.

With “serial, in-line AEX and HIC” we mean that AEX and HIC are serially connected in such a way that the outflow of the AEX device is directly fed into the HIC device, without intermediate storage.

With “flow-through mode” is meant here that the antibodies to be purified pass through the chromatographic device. This contrasts with “capture mode” usually used in antibody purification, wherein the antibodies first bind to the chromatographic material and in a subsequent step are eluted (i.e. released by changing the medium conditions or composition).

In a particular embodiment the method according to the invention involves that the treatments with AEX and HIC are performed as a single unit operation.

With a “single-unit operation’ is meant here that the two serially connected chromatographic devices (AEX and HIC) are used in a single operation step.

Prior to the first ion exchange chromatography step, the cell broth produced in the bioreactor generally will be clarified (i.e. freed from all cellular material, such as whole cells and cell debris).

Also, prior to the first ion exchange chromatography step, a conditioning solution may be added (to the cell broth or to the antibody containing solution freed from the cell material) in order to ensure optimum conditions in terms of pH and conductivity for this first ion exchange step.

With “flow-through fraction” is meant here at least part of the loaded antibody-containing fraction which leaves the chromatographic column without substantially being bound and/or at substantially the same velocity as the elution fluid. Preferably, this fraction is substantially not retained on the column during elution. Hence the conditions are chosen such that not the antibodies but the impurities are bound to the anion exchange material and to the hydrophobic interaction material.

Separation of proteins using subsequent treatment of the protein mixture with anion exchange and hydrophobic interaction chromatography has been disclosed in WO2006/020622. However, in this publication both (AEX and HIC) chromatographic columns are used in binding plus elution mode. Furthermore, this treatment was described as a pre-purification prior to analysis of the protein mixture by 2D electrophoresis. Hence it was a (very) small scale separation.

It has been found that for large scale production purposes the method according to the present invention (with flow-through mode) provides a much faster separation than the prior disclosed method with binding and elution of the desired antibodies.

Advantageously, the separation mixture containing the antibody prior to HIC treatment is supplemented with an adequate amount of lyotropic/kosmotropic salt. The anion of the salt may preferably be selected from the group consisting of phosphate, sulfate, acetate, chloride, bromide, nitrate, chlorate, iodide and thiocyanate ions. The cation of the salt may preferably be selected from the group consisting of ammonium, rubidium, potassium, sodium, lithium, magnesium, calcium and barium ions. Preferred salts are ammonium sulfate, sodium sulfate, potassium sulfate, ammonium phosphate, sodium phosphate, potassium phosphate, potassium chloride and sodium chloride.

Preferably, supplementing the separation mixture with an adequate amount of lyotropic salt is part of the single unit operation e.g. by in-line mixing of the salt in the process stream (e.g. in a mixing chamber) prior to the HIC step.

With “an adequate amount of a lyotropic salt” is meant here sufficient lyotropic salt to cause adsorption of the majority of relevant impurities to the hydrophobic interaction material, but an amount that is low enough not to cause binding or precipitation of the product. For each purification process the optimum amount and preferred type of salt have to be established. In case ammonium sulfate is used, the concentration after in-line mixing will most likely be in between 0.1 and 1.0 M.

AEX treatment according to the invention may take place in an AEX unit which may be embodied by a classical packed bed column containing a resin, a column containing monolith material, a radial column containing suitable chromatographic medium an adsorption membrane unit, or any other chromatographic device known in the art with the appropriate medium and ligands to function as an anion exchanger. In the AEX column the chromatographic material may be present as particulate support material to which strong or weak cationic ligands are attached. The membrane-type anion exchanger consists of a support material in the form of one or more sheets to which strong or weak cationic ligands are attached. The support material may be composed of organic material or inorganic material or a mixture of organic and inorganic material. Suitable organic materials are agarose based media and methacrylate. Suitable inorganic materials are silica, ceramics and metals. A membrane-form anion exchanger may be composed of hydrophilic polyethersulfone containing cationic ligands. Suitable strong cationic ligands are based e.g. on quaternary amine groups. Suitable weak cationic ligands are based on e.g. primary, secondary or tertiary amine groups or any other suitable ligand known in the art.

HIC treatment according to the invention may take place in an HIC unit which may be embodied by a classical column containing a resin, a column based on monolith material, a radial column containing suitable chromatographic medium, an adsorption membrane unit, or any other chromatographic device known in the art with the appropriate ligands to function as a hydrophobic interaction material. In the HIC column the chromatographic material may be present as particulate support material to which hydrophobic ligands are attached. The membrane-like chromatographic device consists of a support material in the form of one or more sheets to which hydrophobic ligands are attached. The support material may be composed of organic material or inorganic material or a mixture of organic and inorganic material. Suitable organic support materials are composed of e.g. hydrophilic carbohydrates (such as cross-linked agarose, cellulose or dextran) or synthetic copolymer materials (such as poly(alkylaspartamide), copolymers of 2-hydroxyethyl methacrylate and ethylene dimethacrylate, or acylated polyamine). Suitable inorganic support materials are e.g. silica, silica, ceramics and metals. A membrane-form HIC may be composed of hydrophilic polyethersulfone containing hydrophobic ligands. Suitable examples of hydrophobic ligands are linear or branched chain alkanes (such as methyl, ethyl, propyl, butyl, pentyl, hexyl, heptyl or octyl), aromatic groups (such as a phenyl group), ethers or polyethers such as polypropylene glycol.

Antibodies which can be purified according to the method of the present invention are antibodies which have an isoelectric pH of 6.0 or higher, preferably 7.0 or higher, more preferably 7.5 or higher. These antibodies can be immunoglobulins of either the G, the A, or the M class. The antibodies can be human, or non-human (such as rodent) or chimeric (e.g. “humanized”) antibodies, or can be subunits of the abovementioned immunoglobulins, or can be hybrid proteins consisting of a immunoglobulin part and a part derived from or identical to another (nonimmunoglobin) protein.

Surprisingly, the antibody material resulting from the combined AEX and HIC treatment generally will have a very high purity (referring to protein content) of at least 98%, preferably at least 99%, more preferably at least 99.9%, even more preferably at least 99.99%.

The anion exchange chromatography step according to the present invention preferably is carried out at neutral or slightly alkaline pH. It will remove the negatively charged impurities like DNA, host cell proteins, protein A (if present), viruses (if present), proteinacous medium components such as insulin and insulin like growth factor (if present).

During the subsequent hydrophobic interaction chromatography step the major remaining large molecular impurities (mainly product aggregates) will be removed, using the property that they are more hydrophobic than the monomeric product and setting the conditions such, that they bind to the chromatographic device while the product flows through.

Subsequently, the highly purified material will, generally, have to be treated by ultrafiltration and diafiltration, in order to remove all residual low molecular weight impurities, to replace the buffer by the final formulation buffer and to adjust the desired final product concentration. This step also assures the removal of the added lyotropic salt.

Furthermore, the highly purified material will, generally, have to be treated also to assure complete removal of potentially present infectious agents, such as viruses and/or prions.

The present invention also relates to a single operational unit comprising both an anion exchange chromatography part (AEX) and a hydrophobic interaction chromatography part (HIC), which are serially connected. This single operational unit further comprises an inlet at the upstream end of the anion exchange chromatography part and an outlet at the downstream end of the hydrophobic interaction chromatography part. This single operational unit also comprises a connection between the anion exchange chromatography part and the hydrophobic interaction chromatography part further comprising an inlet for supply of a lyotropic salt solution to the latter part, hence to the separation mixture.

The liquid flow during the process according to the present invention can be established by any dual pump chromatographic system commercially available, e.g. an ÅKTA explorer (GE), a BIOPROCESS (GE) any dual pump HPLC system or any tailor made device complying with the diagram of FIG. 1. Most of these chromatographic devices are designed to operate a single chromatographic unit (i.e. column or membrane). With a simple adaptation, an extra connection can be made to place the anion exchange after pump A and before the mixing chamber.

FIG. 1 displays the basic configuration. Serial inline connection of two chromatographic devices plus an optional pre-filter in the position as shown in FIG. 1, may lead to undesirable pressure buildup. Therefore, under some conditions extra technical adaptations (e.g. an extra pump after the AEX unit and a pressure reducing device before the AEX unit) may have to be included into this diagram).

### DESCRIPTION OF THE FIGURE

FIG. 1: A single operational unit comprising both an anion exchange chromatography part and a hydrophobic interaction chromatography part. Buffer A is a conditioning and washing buffer suitable for optimum operation of the AEX step. Buffer B contains a lyotropic salt and is mixed in a ratio to the load/buffer A required to obtain optimum conditions for operation of the HIC step. The mixing ratio can be executed using a fixed volumetric mixing flow or can be automatically controlled by a feed back loop, based on e.g. the conductivity output. MC is an optional mixing chamber, which may contain any type of static mixer.

### L=Load

### PA=Pump A

### PB=Pump B

AEX=anion exchange unit
HIC=hydrophobic interaction chromatography unit
pH=pH sensor
σ=conductivity sensor
PF=optional pre-filter

### EXAMPLES

### Materials and Methods

All experiments were carried out using an IgG 1 produced by clone P419 of the human cell-line PER.C6.

The cultivation was carried out in fed-batch, using a chemically defined medium and afterwards the cells were removed by a three step depth filtration filter train ZetaPlus 10M02P, ZetaPlus 60ZA05 and SterAssure PSA020 all from Cuno (3M).

This clarified harvest contained 7.5 g/L IgG and was stored at 2-8° C. First an initial purification by standard Protein A chromatography was carried out using MabSelect (GE) with standard procedures (loading clarified harvest, first wash with 20 mM Tris+150 mM NaCl, second wash with buffer at pH 5.5 and eluting with buffer pH 3.0). In order to find optimized buffer conditions for the subsequent purification, the second wash and elution were carried out with either 100 mM acetate buffer or with 100 mM citrate buffer.

After MabSelect elution, the eluted peak was collected and maintained for 1 hour at pH 3.5. After that, the sample was neutralized to pH 7.4 using 2M Tris pH 9.0 and diluted with demineralized water in order to set the conductivity to 5.0 mS and was filtered over 0.22 p.m.

The material thus obtained was pre-purified IgG either in acetate Tris buffer or in citrate Tris buffer. With this material 3 series of experiments were carried out: 1. to establish optimum conditions for AEX chromatography in flow-through mode (Experiment 1). 2. to establish optimum conditions using HI-chromatography in flow-through mode (Experiment 2). 3. combining both optimized AEX and HIC conditions in one single unit operation experiment (Example 1).

HCP was measured by ELIZA with polyclonal anti-PER.C6 HCP.

Monomeric IgG and aggregate concentrations were determined by size exclusion chromatography (HP-SEC) according to standard procedures.

### Experiment 1

### Establishing Optimum Conditions for Anion Exchange Chromatography in Flow-Through Mode

AEX chromatography in flow-through mode was carried out using mentioned pre-purified IgG either in acetate Tris buffer or in citrate Tris buffer. The following AEX media were tested: Mustang Q coins (0.35 ml) (Pall), Sartobind Q capsule (1 ml), ChromaSorb capsule (0.08 ml) (Millipore) (all membrane adsorbers) and with packed bed column using Poros 50 HQ resin (applied Biosystems) (1 ml packed bed).

All AEX media were run in flow-through using an ÅKTA explorer at 40 bed volumes/hr. Conditioning and washing buffer were either with 100 mM acetate Tris pH 7.4 (for the product runs in acetate buffer) or with 100 mM citrate Tris pH 7.4 (for the product runs in citrate buffer). The amount of product loaded on each AEX medium was 1.5 g/ml membrane or column bed volume.

HCP was measured before and after the chromatography steps. HCP removal is considered as most critical for the AEX chromatographic performance. The log reductions for HCP were 1.9, 1.7, 1.8 and 2.1, respectively, for the before mentioned anion exchangers (all single experiments). Using the citrate matrix, all AEX media performed considerably worse, resulting in an HCP log reduction of 1.2, 0.2. and 1.3 for Mustang Q, Chromasorb and Poros 50 HQ, respectively. These results showed that all AEX chromatographic media tested, were suitable for substantial HCP removal using an acetate buffer and showed approximately comparable HCP log reduction under these conditions.

### Experiment 2

### Establishing Optimum Conditions Using Hydrophobic Interaction Chromatography in Flow-Through Mode

For the HIC step 4 resins were tested: Phenyl Sepharose FF lowsub (GE), Toyopearl PPG 600 (Tosoh), Toyopearl phenyl 600 (Tosoh), Toyopearl butyl 600 (Tosoh).

For these experiments the pre-purified IgG was in 100 mM acetate Tris buffer pH 7.4, conductivity 5.0 mS. In addition, the MabSelect pre-purified IgG containing material was incubated for 40 min at pH 4 and 50° C. in order to increase the amount of aggregates to approximately 20%.

For conditioning and washing, 100 mM acetate Tris buffer pH 7.4, conductivity 5.0 mS was used (buffer A) inline mixed with a certain volume percentage of buffer B. Buffer B contained 2M ammonium sulfate in 100 mM acetate Tris buffer pH 7.4. All resins were tested using inline mixing on volume basis with buffer B during product loading. Several percentage ratios for Load/Buffer A and buffer B were tested for each resin. All column volumes were 1 ml, the flow rate was 100 ml/hr and the amount of IgG in the load was 0.29 g/l and 100 ml was loaded.

Both the load and the flow-through were sampled and analyzed.

Toyopearl phenyl 600, Toyopearl butyl 600 both already at 0% B bound most of the IgG as well as the aggregates. It was therefore concluded that these resins were not suitable for aggregate removal in the flow-through mode using the P419 IgG under the applied conditions.

Both Phenyl Sepharose FF lowsub (not shown), Toyopearl PPG 600 (see Table 1) gave a good aggregate clearance in the flow-through using in-line mixing of the ammonium sulfate containing buffer B at a certain ratio.

TABLE 1
Aggregate clearance using Toyopearl PPG 600 using different volume
ratios of in-line mixing of ammonium sulfate containing buffer B.

| % buffer B | Aggregates (%) | IgG monomer (%) | A²⁸⁰ |
|---|---|---|---|
| Starting material | 19.8 | 79.7 | 0.29 |
| 0 | 20.9 | 78.4 | 0.28 |
| 5 | 17.5 | 81.8 | 0.26 |
| 10 | 7.6 | 91.9 | 0.23 |
| 15 | 1.1 | 98.5 | 0.19 |
| 20 | 0.0 | 99.2 | 0.11 |

### Example 1

### Purification of IgG at Optimized AEX and HIC Conditions in One Single Unit Operation

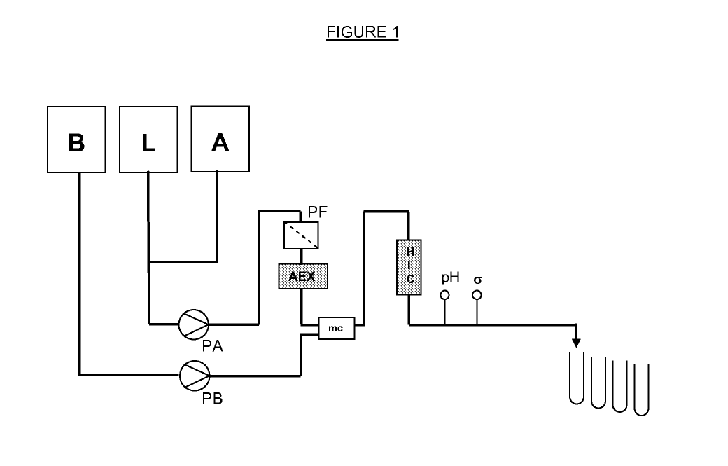

An AEX unit and an HIC unit were serially coupled in-line as depicted in the diagram of FIG. 1. For the AEX a Mustang Q coin was used and for the HIC a column containing 3 ml Toyopearl PPG 600 resin was used.

For resin conditioning before product loading a 100 mM acetate Tris buffer pH 7.4, conductivity 5.0 mS was used (buffer A). Simultaneously, buffer B was mixed in-line at a 22% volume ratio. Buffer B contained 2M ammonium sulfate in 100 mM acetate Tris buffer pH 7.4.

The loading of the pre-purified IgG was started by pumping the IgG at a similar flow as buffer A, while buffer A pumping was stopped. An amount of 362 ml containing 4.37 g IgG was loaded. After completing the loading, the pump was switched back to buffer A, in order to recover all product from the system. After that the HIC unit was stripped by stopping the in-line mixing of buffer B and hence use 100% buffer A (separately collected). During the whole run the flow over the HIC was 185 ml/hr. The total time (including conditioning washing and stripping) was 3.5 hours. Both the load and the flow-through were analyzed for IgG aggregate ratio, DNA content, HCP content and protein (product) content (A²⁸⁰). The HCP reduction was >log 2.3 (the amount of HCP in the flow-through was below LoD). The amount of aggregate was 5.8% in the load and was 1.2% in the flow-through. The overall product recovery based on A²⁸⁰ was 86.7% without stripping and 90.1% including the stripping.

### ABBREVIATIONS USED

### A²⁸⁰ (Light) Absorption at 280 nm

AEX Anion Exchange chromatography
BHK cells Baby Hamster Kidney cells
CHO cells Chinese Hamster Ovary cells

### EBA Expanded Bed Adsorption

### HCP Host Cell Protein

### HIC Hydrophobic Interaction Chromatography

### HPLC High Pressure Liquid Chromatography

### IgG Immunoglobulin G

### LoD Limit of Detection

### TFF Tangential Flow Filtration

Tris tris(hydroxymethyl)methylamine

1. Method for the purification of antibodies from a protein mixture produced in a bioreactor, at least comprising the steps of intermediate purification and polishing, wherein the intermediate purification and polishing steps comprise serial in-line anion exchange chromatography (AEX), yielding as a flow-through fraction a separation mixture, followed by hydrophobic interaction chromatography (HIC) yielding as a flow through fraction a purified antibody preparation, and wherein the purified antibody preparation is subjected to at least one further purification step.

2. Method according to claim 1 wherein anion exchange chromatography and hydrophobic interaction chromatography take place in two separate devices which are serially connected.

3. Method according to claim 1 wherein the serial in-line AEX and HIC are performed as a single unit operation.

4. Method according to claim 1 wherein the separation mixture prior to HIC is supplemented with an adequate amount of lyotropic salt.

5. Method according to claim 1 wherein the separation mixture prior to HIC is supplemented with an adequate amount of ammonium sulfate, sodium sulfate, potassium sulfate, ammonium phosphate, sodium phosphate, potassium phosphate, potassium chloride and sodium chloride.

6. A single operational unit which can be used in a method according to claim 1 comprising both an anion exchange chromatography part and a hydrophobic interaction chromatography part, which are serially connected, wherein the outlet of the anion exchange chromatography part is connected to the inlet of the hydrophobic interaction chromatography part, wherein the unit comprises an inlet at the upstream end of the anion exchange chromatography part and an outlet at the downstream end of the hydrophobic interaction chromatography part and wherein the unit also comprises an inlet between the anion exchange chromatography part and the hydrophobic interaction chromatography part.
